(19) United States
(12) Patent Application Publication
(10) Pub. No.: US 2016/0151214 A1
(43) Pub. Date: Jun. 2, 2016

(54) ISOLATING STRUCTURE FOR EXTERNAL SKELETAL FIXATOR

(71) Applicant: METAL INDUSTRIES RESEARCH & DEVELOPMENT CENTRE, KAOHSIUNG CITY (TW)

(72) Inventors: CHIH-HAO CHANG, TAIPEI CITY (TW); MENG-HSIU WU, KAOHSIUNG CITY (TW); SHIH-HUA HUANG, KAOHSIUNG CITY (TW); BING-HENG LEE, NEW TAIPEI CITY (TW)

(21) Appl. No.: 14/953,071

(22) Filed: Nov. 27, 2015

(30) Foreign Application Priority Data
Nov. 27, 2014 (TW) 103141208

(51) Int. Cl. A61F 15/00; A61F 13/00

(52) U.S. Cl. CPC A61F 15/008; A61F 13/00004

(57) ABSTRACT

The present invention relates to an external skeletal fixator, which comprises a fixing member and a stereoscopic hood. The fixing member is disposed on a first end surface of the stereoscopic hood. The diameter of a second end surface of the stereoscopic hood is greater than that of the first end surface of the stereoscopic hood. When the stereoscopic hood is disposed to a pin passing through a skin tissue and a bone tissue, the second end surface of the stereoscopic hood is against the skin tissue surrounding a pin-skin interface and thus covering the stereoscopic hood over the pin-skin interface. The first end surface and a sidewall of the stereoscopic hood do not contact the pin-skin interface and the skin tissue around. Thereby, the contact area between the isolating structure and the pin-skin interface is reduced and the isolating structure blocks external germs from entering via the pin-skin interface.

### FIELD OF THE INVENTION

The present invention relates generally to an external skeletal fixator, and particularly to an isolating structure for external skeletal fixator.

### BACKGROUND OF THE INVENTION

An external skeletal fixator is to place steel pins at proximal and distal fracture fragments. It is a special apparatus made by metallic or high-strength nonmetallic materials for connecting pins exposed to the skin. In general, an external skeletal fixator is applicable to second- to fourth-degree open fractures, fractures accompanying severe burns, partially unstable fractures, multiple fractures, infectious fractures, fractures accompanying soft-tissue defects and requiring neurorrhaphy or angiorrhaphy, or reconstruction surgeries such as flap transplantation or grafting.

The normal installation method for external skeletal fixator is first to reposition the fractured bones with the aid of an X-ray machine. Then the external skeletal fixator is implanted and fixed for providing stable support to the fractured bones. The external skeletal fixator can have fixation, pressing, and traction functions and thus achieving the purposes of treating fractures, correcting bone and joint deformity, and extremity extension. In addition, the standard treatment for severe fractures is to adopt external skeletal fixators.

Nonetheless, the pin-skin interfaces of an external skeletal fixator are possibly to be infected. In order to prevent infections at the pin-skin interfaces, attention should be paid to wound care (sterilization and change of medicine). Currently, carers will remove the swab or silica gel covering the wound. They judge the conditions of pin-skin interfaces, for example, secretions, exudations, quantity, color, smell, and use consumables such as cotton or swabs together with alcohol to clean the wound. When the wound has secretions, after sterilization, it is required to cover pin-skin interfaces using sterile swabs or Y-type swabs and fix them at the ends using ventilative tapes. Nonetheless, the nursing methods are labor intensive and complicated. Besides, a normal cycle for wound care takes 3 to 7 days. By using only tapes to fix swabs or silica gel at the pin-skin interface, the fixation will be insufficient and attachment to the wound is limited. Without long-standing fixation, the risk for postoperative infections is increased.

Accordingly, by using the external skeletal fixator according to the prior art, the nursing process is complicated. Besides, the problems of insufficient fixation and limited attachment to the wound will occur by using tapes to fix swabs over the wound. Thereby, how to simplify the caring process and reduce the risk for postoperative infections have become the major subjects in this field.

### SUMMARY

An objective of the present invention is to provide an isolating structure for external skeletal fixator, which reduces the contact area of a pin-skin interface, where a pin passes through a skin tissue, and isolating external germs from entering. Thereby, germ breeding and infections can be prevented effectively at the pin-skin interface and hence reducing postoperative infections effectively.

The present invention provides an isolating structure for external skeletal fixator, which comprises a fixing member and a stereoscopic hood. The stereoscopic hood includes a first end surface, a second end surface, and a sidewall. The sidewall is located between the first end surface and the second end surface of the stereoscopic hood. The fixing member is disposed on the first end surface. The diameter of the second end surface of the stereoscopic hood is greater than that of the first end surface of the stereoscopic hood.

### BRIEF DESCRIPTION OF THE DRAWINGS

FIG. 1 shows a schematic diagram of the isolating structure according to the first embodiment of the present invention;

FIG. 2 shows an assembly diagram of the isolating structure according to the first embodiment of the present invention;

FIG. 3 shows a cross-sectional view of the isolating structure according to the first embodiment of the present invention;

FIG. 4 shows a usage status diagram of the isolating structure according to the first embodiment of the present invention;

FIG. 5 shows a schematic diagram of the isolating structure according to the second embodiment of the present invention;

FIG. 6 shows a schematic diagram of the isolating structure according to the third embodiment of the present invention; and

FIG. 7A and FIG. 7B show other usage status diagrams of the isolating structure according to the first embodiment of the present invention.

### DETAILED DESCRIPTION

In order to make the structure and characteristics as well as the effectiveness of the present invention to be further understood and recognized, the detailed description of the present invention is provided as follows along with embodiments and accompanying figures.

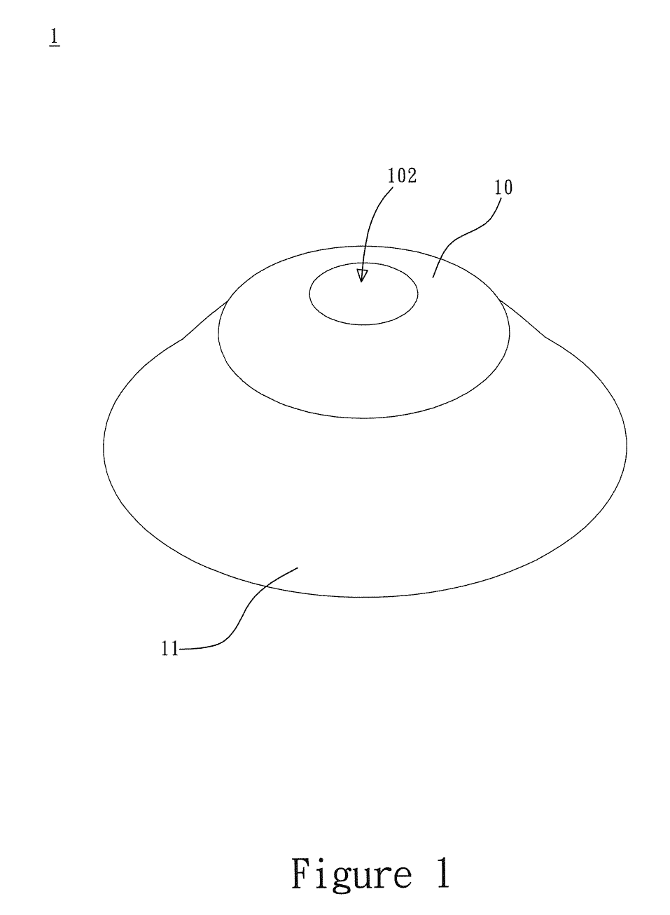

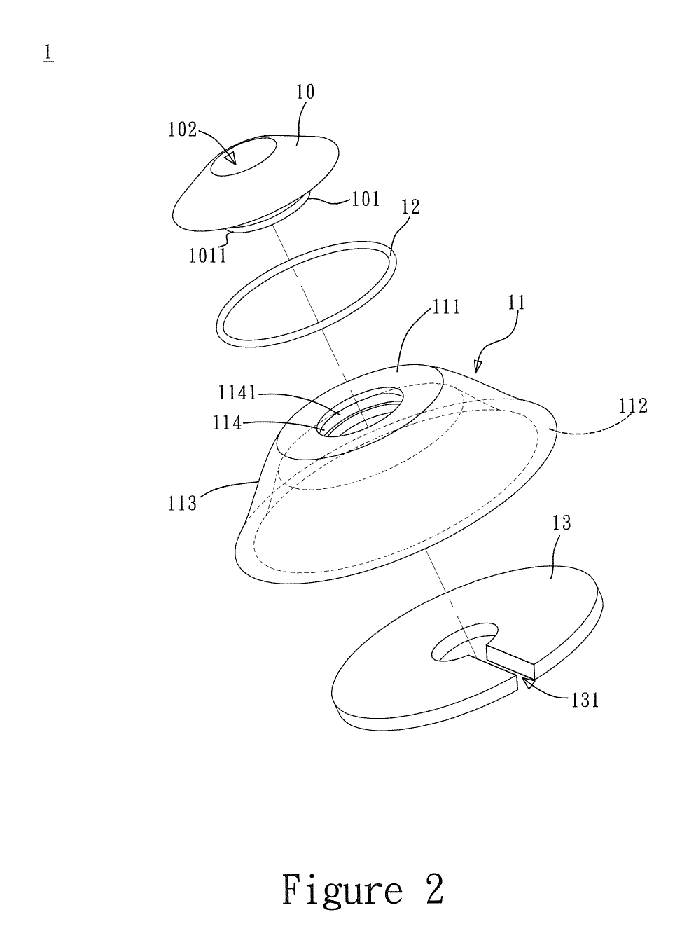

Please refer to FIG. 1 and FIG. 2, which show a schematic diagram and an assembly diagram of the isolating structure according the first embodiment of the present invention. As shown in the figures, the present embodiment provides an isolating structure 1 for external skeletal fixator. The isolating structure 1 comprises a fixing member 10 and a stereoscopic hood 11. The fixing member 10 includes a first connecting part 101 and a hole 102. The hole 102 penetrates the first connecting part 101. The stereoscopic hood 11 includes a first end surface 111, a second end surface 112, and a sidewall 113. The sidewall 113 is located between the first end surface 111 and the second end surface 112. Besides, the diameter of the second end surface 112 of the stereoscopic hood 11 is greater than that of the first end surface 111 of the stereoscopic hood 11. The stereoscopic hood 11 is coniform. The first end surface 111 includes a second connecting part 114 connecting with the first connecting part 101 of the fixing member 10 such that the fixing member 10 is disposed on the first end surface 111 of the stereoscopic hood 11.

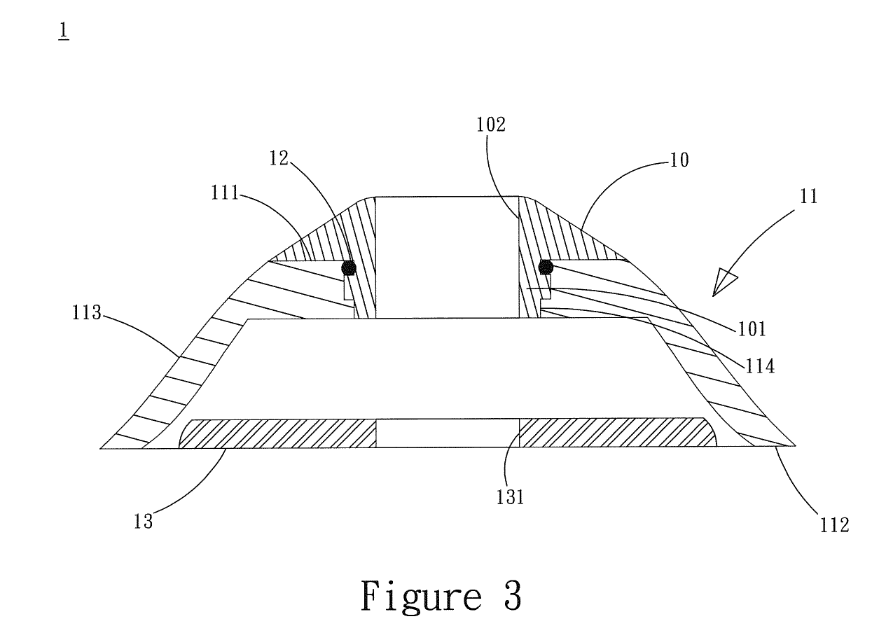

The first connecting part 101 of the fixing member 10 according to the present embodiment is a pillar having a first thread 1011. The second connecting part 114 of the stereoscopic hood 11 is a hole having a second thread 1141 on the sidewall and corresponding to the first thread 1011 of the first connecting part 101. The first thread 1011 meshes the second thread 1141. According to the present embodiment, the first connecting part 101 and the second connecting part 114 are connected via threads. Alternatively, they can be connected to each other by wedging. The details will not be described here. Please refer to FIG. 3, which shows a cross-sectional view of the isolating structure according to the first embodiment of the present invention. As shown in the figure, a sealing member 12 is disposed between the first connecting part 101 of the fixing member 10 according to the present embodiment and the second connecting part 114 on the first end surface 111 of the stereoscopic hood 11. The sealing member 12 is an elastic ring used for filling the gap between the first and second connecting parts 101, 114 and enabling tight attachment between the stereoscopic hood 11 and the fixing member 10.

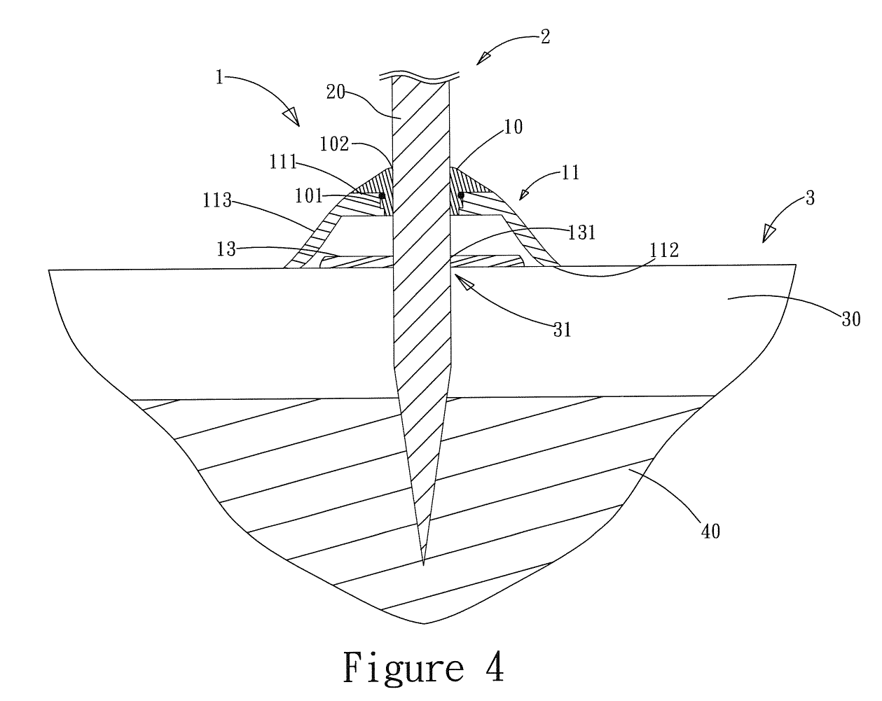

Please refer to FIG. 4, which shows a usage status diagram of the isolating structure according to the first embodiment of the present invention. As shown in the figure, when the isolating structure 1 is applied to the external skeletal fixator 2, the external skeletal fixator 2 is first fixed to a human body 3 The external skeletal fixator 2 includes one or more pin 20, which passes through a skin tissue 30 and a bone tissue 40 of the human body 3 for fixing the bone tissue 40. The isolating structure 1 is disposed at the pin 20. The end of the pin 20 not passing the skin and bone tissues 30, 31 enters the second end surface 112 of the stereoscopic hood 11 of the isolating structure 1 and passes through the hole 102 of the fixing member 10. The isolating structure 1 moves towards the human body 3 along the pin 20. After the pin 20 passes through a pin-skin interface 31 of the skin tissue 30, and the skin tissue 30 surrounding the pin-skin interface 31 and the isolating structure 1 are sterilized, The second end surface 112 of the stereoscopic hood 11 of the isolating structure 1 is against the skin tissue 30 surrounding the pin-skin interface 31, making the stereoscopic hood 11 to cover the pin-skin interface 31 and the skin tissue 30 around. Because the stereoscopic hood 11 is stereoscopic and the adopted material is supportive, it is not deformable without external force. The stereoscopic hood 11 covers the skin tissue 30 by physical pressing. In addition, the first end surface 111 and sidewall 113 of the stereoscopic hood 11 do not contact with the pin-skin interface 31 and the skin tissue 31 around for reducing the contact area between the stereoscopic hood 11 and the pin-skin interface 31, and the skin tissue 30 around. Thereby, the pin-skin interface 31 and the skin tissue 30 around can be kept dry and sterile, avoiding germ breeding and infection conditions at the pin-skin interface 31. Furthermore, the material of the stereoscopic hood 11 according to the present embodiment is silicon-polymer material. As the stereoscopic hood 11 covers the skin tissue 30 by physical pressing, the second end surface 112 of the stereoscopic hood 11 can attach to the skin tissue 30, and hence reinforcing sealing between the stereoscopic hood 11 and the skin tissue 30. Entrance of external germs from the gap between the stereoscopic hood 11 and the skin tissue 30 into the isolating structure 1 can be prevented.

The fixator 10 according to the present embodiment is made by silicon-polymer. When the pin 20 passes through the hole 102 of the fixing member 10, the sidewall of the hole 102 attaches tightly to the periphery of the pin 20, making no gap between the hole 102 and the pin 20 for external germs to enter and thus maintaining sterile condition inside the stereoscopic hold 11. In addition, when the isolating structure 1 moves along the pin 20, it stops moving as the second end surface 112 of the stereoscopic hood 11 is against the skin tissue 30. Then the fixing member 10 fixes the isolating structure 1 at the pin 20 by tight attachment of the sidewall of the hole 102 to the periphery of the pin 20 and thereby covering the stereoscopic hood 11 on the skin tissue 30. The diameter of the hole 102 as described above depends on the diameter of the pin 20. The diameter of the hole 102 can be smaller than to equal to the diameter of the pin 20. Besides, the material of the fixing member 10 owns the features of shape memory and self-healing. Consequently, the fixing member 10 can be reused, reducing waste of medical resources.

The ventilation of the stereoscopic hood 11 can be selected to match the condition of the pin-skin 31 interface. If the stereoscopic hood 11 is not ventilative, external germs can be blocked completely from the isolating structure 1 and preventing germ breeding and deterioration caused by entering of external germ into the isolating structure 1.

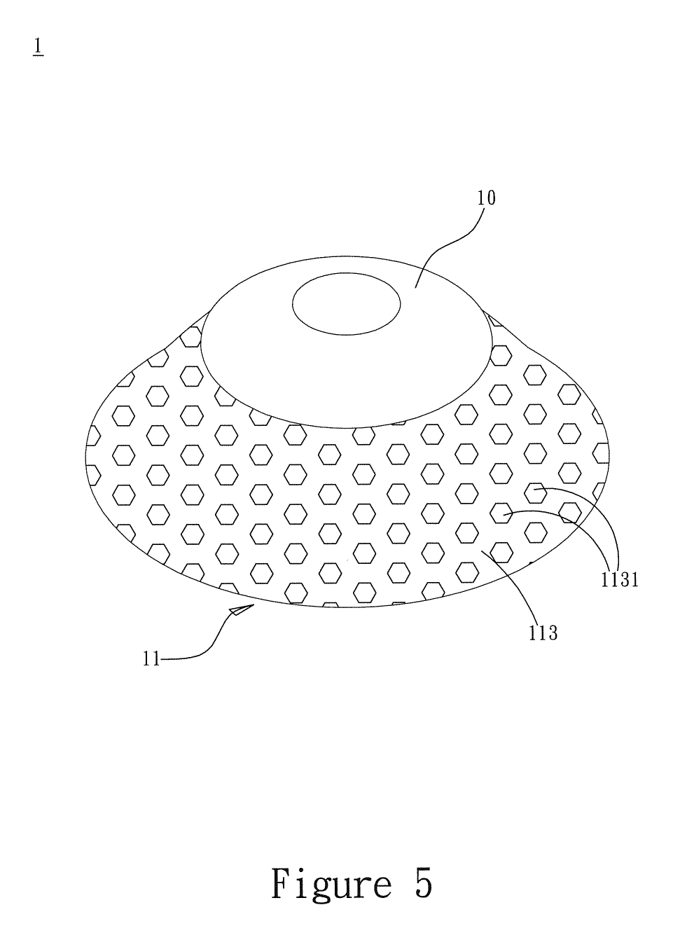

Please refer to FIG. 5, which shows a schematic diagram of the isolating structure according to the second embodiment of the present invention. As shown in the figure, if the stereoscopic hood 11 is highly ventilative, the moisture inside the isolating structure 1 can be expelled and keeping the pin-skin interface 31 dry and avoiding germs breeding. According to the present embodiment, the sidewall 113 of the stereoscopic hood 11 can includes a plurality of micro holes 1131. The shape of the plurality of micro holes 1131 can be circular or honeycomb-shaped. Thereby, the stereoscopic hood 11 can be highly ventilative. The plurality of micro holes 1131 are smaller than external dusts and germs and thereby keeping them from entering the isolating structure 1. The moisture inside the isolating structure 1 can be expelled from the stereoscopic hood 11 and enabling the stereoscopic hood 11 to own the feature of unidirectional ventilation. The above example of enabling the stereoscopic hood 11 to be highly ventilative by forming the plurality of micro hole 1131 on the sidewall 113 is only and embodiment of the present invention. There are still other ways to do so. The details will not be described here.

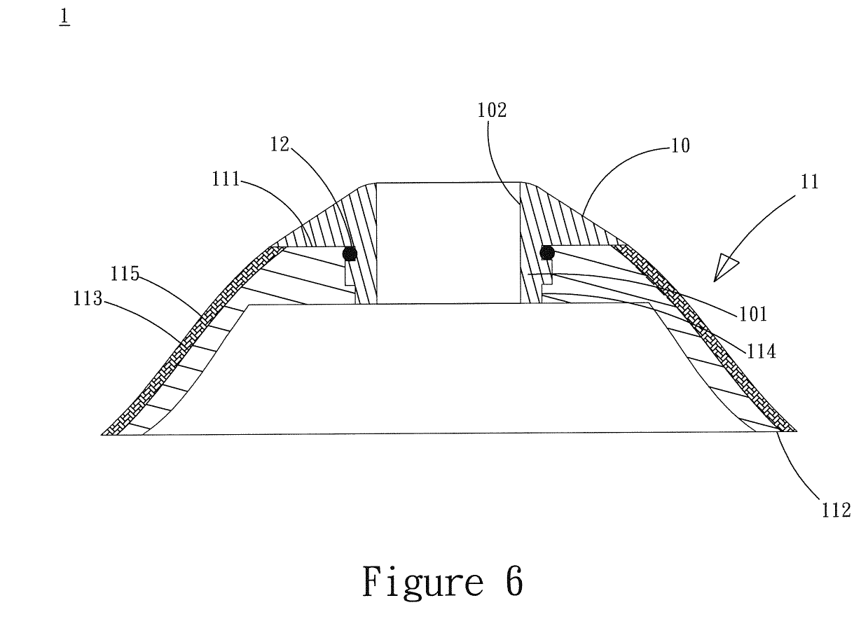

FIG. 6 shows a schematic diagram of the isolating structure according to the third embodiment of the present invention. As shown in the figure, the sidewall 113 according to the present embodiment can further include a ventilative layer 115. The material of the ventilative layer 115 is polyester and mainly woven by polyester fibers. According to the present embodiment, the ventilative layer 115 can be disposed on the outer surface of the sidewall 113. Of course, the ventilative layer 115 can be also disposed on the inner surface of the sidewall 113. Moreover, static agent can be added to the outer surface of the stereoscopic hood 11 for adding static charges thereto. Thereby, when external germs are entering the stereoscopic hood 11, they are attracted by the static charges outside the stereoscopic hood 11 and thus blocking them from entering the isolating structure 1. Accordingly, deterioration of the pin-skin interface 31 due to infection by external germs can be prevented.

The isolating structure 1 according to the present embodiment further comprises a dressing 13 attached to the pin-skin interface 31 for reducing inflammatory reactions and facilitating healing of the pin-skin interface 31. The dressing 13 includes an opening 131. When the dressing 13 is attached to the pin-skin interface 31, the opening 131 attaches and circumvents the pin 20. Thereby, the dressing 13 can attach to the skin tissue 30 along the periphery of the pin 20. The stereoscopic hood 11 according to the present embodiment does not contact the dressing 13. Thereby, no germ-breeding problem will occur.

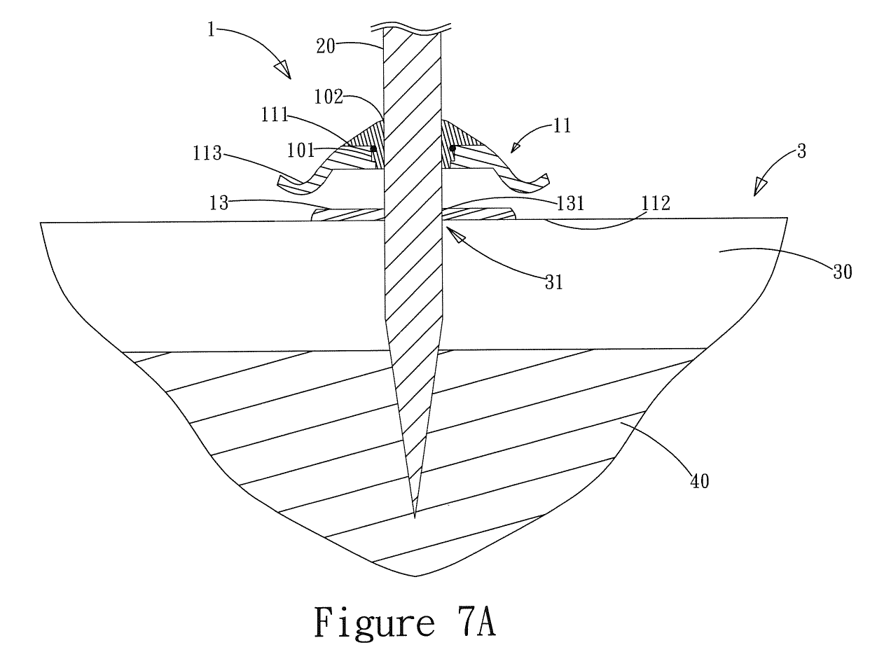

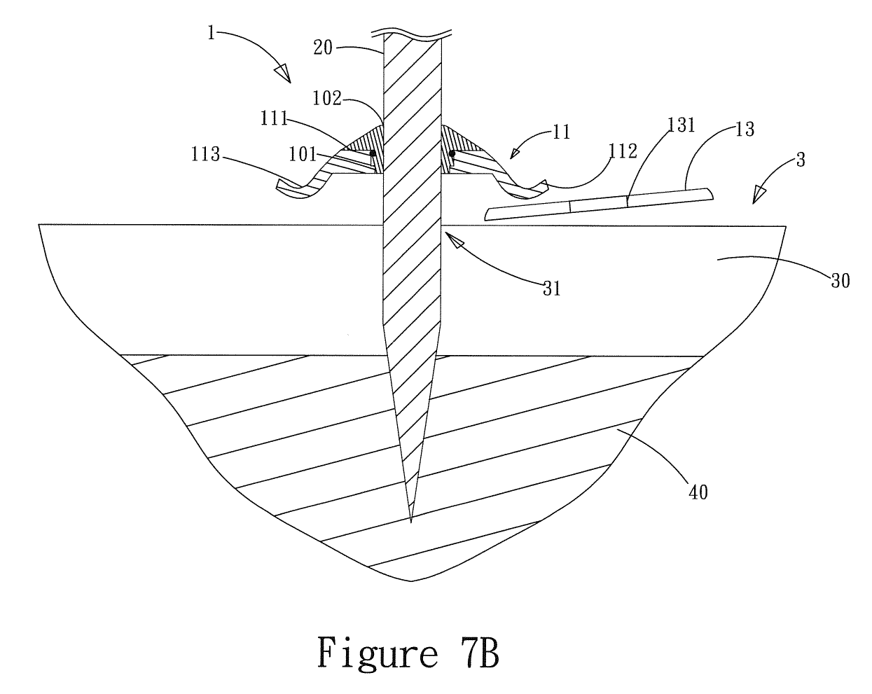

FIG. 7A and FIG. 7B show other usage status diagrams of the isolating structure according to the first embodiment of the present invention. As shown in the figures, when a user or carer provides care service to the pin-skin interface 31, all he/she needs to do is to turn the sidewall 113 of the stereoscopic hood 11 outwards. Because the material of the stereoscopic hood 11 is tenacious, the stereoscopic hood 11 can be fixed in the outward-turned condition. The user or carer can take out the existing dressing 13 directly and sterilize the pin-skin interface 31 and the skin tissue 30 around. Then a new dressing 13 can be placed at the pin-skin interface 31. After the above care activities are finished, the user or carer can dismiss the outward-turned of the stereoscopic hood 11 and the second end surface 112 of the stereoscopic hood 11 can move downward automatically and contact against the skin tissue 30. According to the above description, during the caring process, the stereoscopic hood 11 can be fixed in the outward-turned condition automatically, thereby the user or carer can concentrate on caring the pin-skin interface 31 and improving convenience for caring. In addition, during the caring process, contact of the stereoscopic hood 11 with the pin-skin interface 31 can be avoided as well, preventing the germ-breeding problem.

To sum up, the present invention provides an insolating structure for external skeletal fixator. The inside of the isolating structure does not contact the pin-skin interface. Thereby, the germ-breeding problem caused by long-term contact between the isolating structure and the pin-skin interface can be prevented. In addition, the isolating structure can prevent entering of external germs, enabling a sterile condition between the isolating structure and the skin tissue and thus preventing the germ-breeding problem. The isolating structure according to the present invention can be reused and hence reducing waste of medical resources.

Accordingly, the present invention conforms to the legal requirements owing to its novelty, nonobviousness, and utility. However, the foregoing description is only embodiments of the present invention, not used to limit the scope and range of the present invention. Those equivalent changes or modifications made according to the shape, structure, feature, or spirit described in the claims of the present invention are included in the appended claims of the present invention.

What is claimed is:

1. An isolating structure for external skeletal fixator, comprising:
a fixing member; and
a stereoscopic hood, having a first end surface, a second end surface, and a sidewall, said sidewall located between said first end surface and said second end surface of said stereoscopic hood, said fixing member disposed on said first end surface, and the diameter of said second end surface of said stereoscopic hood greater than the diameter of said first end surface of said stereoscopic hood.

2. The isolating structure for external skeletal fixator of claim 1, wherein the shape of said stereoscopic hood is coniform.

3. The isolating structure for external skeletal fixator of claim 1, wherein said sidewall of said stereoscopic hood includes a plurality of micro holes and a ventilative layer.

4. The isolating structure for external skeletal fixator of claim 3, wherein the material of said ventilative layer is polyester.

5. The isolating structure for external skeletal fixator of claim 1, further comprising a dressing located inside said stereoscopic hood and including an opening.

6. The isolating structure for external skeletal fixator of claim 1, wherein said fixing member includes a first connecting part; said stereoscopic hood includes a second connecting part corresponding to said first connecting part; said second connecting part is located on said first end surface; and when said fixing member is disposed on said first end surface of said stereoscopic hood, said first connecting part is connected with said second connecting part.

7. The isolating structure for external skeletal fixator of claim 6, wherein said first connecting part includes a first thread and said second connecting part includes a second thread; and when said first connecting is connected with said second connecting part, said first thread meshes said second thread.

8. The isolating structure for external skeletal fixator of claim 6, wherein a sealing member is further disposed between said first connecting part and said second connecting part.

9. The isolating structure for external skeletal fixator of claim 1, wherein said fixing member includes a hole.

10. The isolating structure for external skeletal fixator of claim 1, wherein the material of said fixing member or/and said stereoscopic hood is silicon-polymer.
